(19) United States
(12) Patent Application Publication
(10) Pub. No.: US 2020/0352771 A1
(43) Pub. Date: Nov. 12, 2020

(54) ORTHOTIC FOOT PART

(71) Applicant: Ottobock SE & Co. KGaA, Duderstadt (DE)

(72) Inventors: Julia BIALOWONS, Göttingen (DE); Klaus LIDOLT, Duderstadt (DE); Johan Fredrik FLOOD, Katrineholm (SE); Guido KAHLMEYER, Siemerode (DE)

(73) Assignee: Ottobock SE & Co. KGaA, Duderstadt (DE)

(21) Appl. No.: 15/733,115

(22) PCT Filed: Nov. 14, 2018
(86) PCT No.: PCT/EP2018/081252

§ 371 Date: May 19, 2020

(30) Foreign Application Priority Data
Nov. 24, 2017 (DE) 10 2017 127 889.3

(51) Int. Cl. A61F 5/01

(52) U.S. Cl. CPC A61F 5/0127; A61F 2005/0179

(57) ABSTRACT

An orthotic foot part having a foot plate with a heel region and a front foot region, and at least one strut extending in a proximal direction from the foot plate. The foot plate has a foot plate contour formed of superposed, stylized contours of a right foot and a left foot.

The invention relates to an orthotic foot part having a foot plate with a heel region and a front foot region and at least one strut extending in a proximal direction from the foot plate.

These foot parts are arranged on orthoses of a lower extremity in order to allow the foot to be placed on them. At the level of the natural ankle joint, the orthotic foot parts can have a joint or can have a receptacle for a joint device, thereby permitting a more or less free pivoting movement of the orthotic foot part, together with the foot, relative to the lower leg and to a lower-leg part of an orthosis. The orthotic foot part can be spring-loaded, for example in order to avoid unintended plantar flexion in cases of complete or partial paralysis. It is also possible that a drive is assigned to the orthotic joint, for example in order to perform, or at least assist, a plantar flexion and dorsal flexion under sensor control. In addition, there are orthoses of the lower extremity with a foot plate in which a more or less rigid strut extends perpendicularly from the foot plate along the lower leg, in order to hold the foot in a predetermined position with respect to the lower leg. The strut can be designed to be resilient, in order to be able to take up the impact at heel strike during walking and to store this as deformation energy, to facilitate roll-over, and then to provide a desired elastic resistance during dorsal flexion all the way through to toe-off. In the final stage of the stance phase, the energy stored during the roll-over is converted again into a plantar flexion movement, thereby assisting a natural gait.

The foot plates are either prepared individually for a particular patient or are prefabricated. A problem is the fact that different foot plates or entire orthoses have to be produced for different shoe sizes. In addition, different orthoses have to be kept in stock for right and left feet, which increases the costs of production and storage of the different orthoses.

The object of the present invention is to make available an orthosis of a lower extremity that permits cost-effective manufacture and cost-effective management of the patient.

According to the invention, this object is achieved by an orthotic foot part having the features of the main claim. Advantageous embodiments and developments of the invention are set forth in the dependent claims, the description and the figures.

In the orthotic foot part according to the invention having a foot plate with a heel region and a front foot region and at least one strut extending from the foot plate in a proximal direction, i.e. in a direction of a lower leg, provision is made that the foot plate has a foot plate contour formed of superposed, stylized contours of a right and left foot. Instead of separately producing an orthosis or an orthotic foot part for a right foot and a left foot, provision is made according to the invention that, instead of a foot plate contour with stylized contours of a right or left foot, both contours are superposed so as to provide one foot plate contour on which both feet can be placed, without losing the required support provided by the foot plate. If two stylized contours of a right foot and a left foot or of a foot plate for a left foot and a right foot are superposed in the heel region, where the contours of the foot plates of a left and a right foot correspond, this results in the front foot region having a flared foot plate contour which widens to the left and right along the longitudinal axis and which can be easily adapted to the particular purpose of use. The foot plate regions not required, which protrude beyond the actual contour of the foot that is set down, are removed, for example cut off or ground off, thereby permitting an individual adaptation to the particular foot to be treated or to the particular shoe in which the orthosis with the orthotic foot part is intended to be worn. An orthopedic technician no longer has to keep a large number of orthoses or orthotic foot parts in stock in order to treat patients who have a variety of foot sizes and foot shapes, on the right foot and on the left foot, and instead, with the orthotic foot part according to the invention, it suffices to keep in stock just a single sample, with which an orthosis of a lower extremity can be produced or at least adapted by customization to the particular patient.

The foot plate contour is preferably designed to be axially symmetrical with respect to a longitudinal axis, which extends from the heel region to the front foot region. The longitudinal axis preferably extends along a midline along the longitudinal direction of the foot from the heel region, approximately at the level of the natural ankle joint, forward in the direction of the front foot region. This corresponds to the orientation from the heel into the natural walking direction. The axially symmetrical design makes manufacture particularly easy, since the mold for the orthotic foot part can be easily produced.

In a development of the invention, a recess oriented in the direction of the heel region is formed in a front edge of the foot plate, i.e. at the front end of the foot plate contour, which recess can have a deepest point or, alternatively, a point directed farthest in the direction of the heel region. The front edge of the foot plate preferably has an undulating or rounded contour, such that two projections to both sides of the recess extend symmetrically with respect to a line of symmetry, which also at the same time forms the longitudinal axis of the orthotic foot part. These projections correspond to the design of a conventional shoe sole or foot contour, but without forming a tip directed toward the heel region.

Markings which represent different foot sizes, both for a right and a left foot, can be arranged on the foot plate. By virtue of the foot plate being configured in the intended maximum size, it is possible, with just one foot plate, to be able to provide for all foot sizes, both for a right and a left foot. Alternatively, it is possible to provide different size groups, for example small, medium and large, since feet of different sizes can also have different foot widths and in particular also different ankle widths, such that a support of a lower-leg part on the lower leg can be achieved with a precise fit using grouped sizes of foot plates.

Markings which represent different foot sizes can also be arranged in the heel region, such that it is possible to adjust the heel length and the distance of the rear end of the foot plate contour from the strut which, in the metatarsal region, the ankle region or just behind the ankle, extends upward from the foot plate and along the lower leg.

The markings can either be arranged purely as color markings on the top or underside or can be formed on the underside of the foot plate as depressions or grooves, such that shortening and adaptation to the right and/or left foot can be made easier, since it is only the material extending beyond the groove best matching the foot to be treated that has to be removed. For this purpose, the foot plate is cut along the selected groove, and the material beyond the groove is removed. The arrangement of grooves or recesses on the underside of the foot plate means that the wearing comfort experienced by the user is not affected, since a closed surface of the foot plate is ready for the sole of the foot.

The strut can be designed as a spring element and can allow a resilient movement particularly in the anterior-posterior direction. In principle, it is also possible to allow a medial-lateral spring movement, so as to be able to take up and forward corresponding forces or moments.

In a development of the invention, provision is made that two struts are arranged on the foot plate, one strut being arranged medially and one strut being arranged laterally on the foot plate. It is thus possible to guide two rails medially and laterally along the lower leg or else, by removing one of the struts in the case of a unilateral arrangement of a rail, to make the desired adaptation to the treatment required in each particular case. By the arrangement of two struts medially and laterally on the foot plate, for example in the metatarsal region or in the region of the natural ankle, in conjunction with a universal embodiment of the foot plate contour for use for a left foot and right foot, it is possible, both for a left foot and right foot, to provide medial or lateral guiding of the strut or of a lower-leg rail fastened to the strut. Otherwise, at least four orthoses or orthotic foot parts would be needed to treat one individual, each of them in the respectively matching foot size.

The at least one strut can be formed integrally on the foot plate, such that a one-piece embodiment of the foot plate together with the strut can be realized.

In a novel embodiment providing an alternative to a one-piece design, provision is made that the strut is designed as a U-shaped or L-shaped bracket, which is secured to the foot plate. The securing is in particular reversible, such that the bracket is fastened to the foot plate in an adjustable and lockable manner. In a U-shaped design, two brackets protrude laterally from the foot plate in the proximal direction and, for example, can be mounted in a rotatable and lockable manner about a fastening point. The fastening point is preferably located centrally on the line of symmetry or midline of the foot plate, in the region of the plantar arch, and allows the struts to pivot about an axis perpendicular to the main plane of the foot plate. It is thereby possible to align the struts with the prominences of the ankle and thus to obtain an orientation of an ankle joint axis in a pivotable embodiment of the lower-leg orthosis.

A receptacle for a joint can be formed on the strut, for example a bore or a plug-in device. A receptacle for a rail can be formed at the proximal end of the strut, such that a joint device is formed there directly. In principle, it is also possible that the receptacle is designed as a rail, which can be inserted into and secured in a rail box of a separate joint. A lower-leg rail is then arranged on the other side of the joint device, or a lower-leg shell which is designed to bear on a lower leg. Alternatively, a support element for a shin or calf in the applied state of the foot orthosis can be arranged on the strut, for example in order to form a dorsiflexion assist orthosis without a joint device. The support element can either be formed in one piece with the strut or can be fastened to the strut. The fastening can be reversible, such that different sizes of supports can be coupled to the respective strut.

A device for securing to a lower extremity can be arranged on the foot plate and/or the strut, for example a strap, a belt or a buckle.

Illustrative embodiments of the invention are explained in more detail below with reference to the accompanying figures, in which:

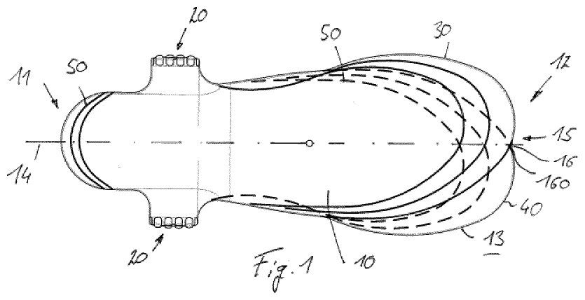

FIG. 1 shows a bottom view of a foot plate,

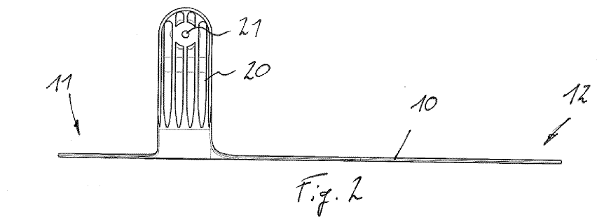

FIG. 2 shows a side view of a foot plate,

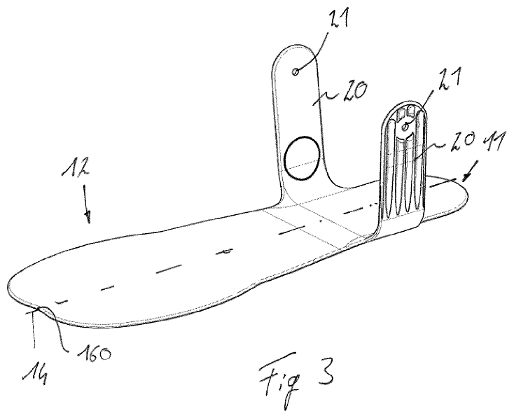

FIG. 3 shows a perspective view of a foot plate with integrally formed struts,

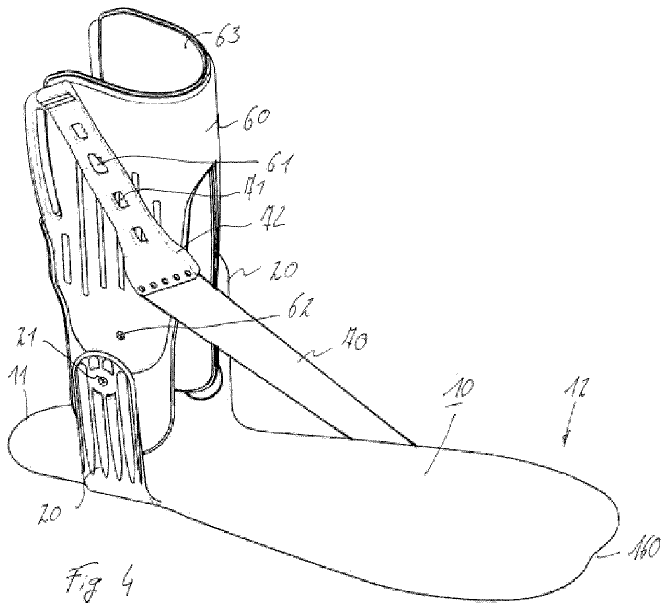

FIG. 4 shows a perspective view of a foot plate with lower-leg support and correcting strap,

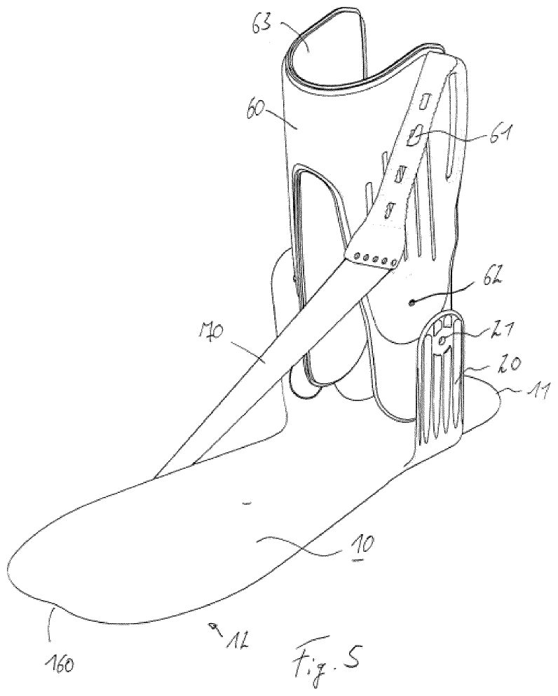

FIG. 5 shows a variant of FIG. 4, and

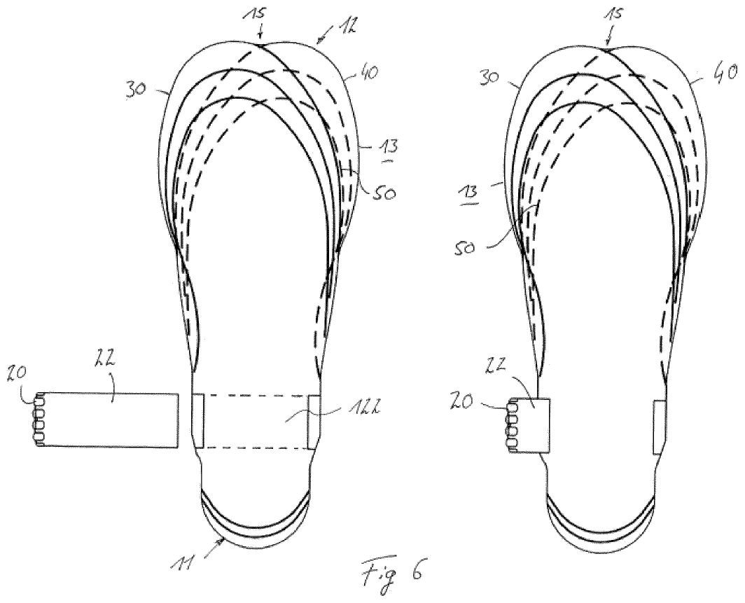

FIG. 6 shows two bottom views of a modular variant of the foot plate.

FIG. 1 shows a bottom view of a foot plate 10 of an orthotic foot part with a heel region 11 and a front foot region 12. In the region of the position of a natural ankle joint, struts 20 are arranged medially and laterally with respect to the foot plate 10, said struts 20 extending into the plane of the drawing, i.e. in the direction of a lower leg of an orthosis user, seen in the proximal direction from the foot plate. The foot plate 10 has a foot plate contour 13 formed of superposed contours 30, 40 of a stylized left and right foot. In the heel region 11, the superpositioning of conventional foot plates or shoe soles results in a covering in which the posterior ankle region is rounded and two substantially parallel side walls extend along the longitudinal extent of the foot in the direction of the front foot region 12. Starting from the position of the natural ankle joint, approximately at the level of the two struts 20, the plantar arch and the configuration of the contact face of a natural foot sole result, in the case of a right foot, in a shape of the foot plate contour which describes an arc extending laterally outward from the ankle region, reaches its most forward point medially with respect to the longitudinal axis 14 or the midline of the foot, in order then to pivot medially inward with a further arc. The medially widest extent is generally in the region of the first metatarsophalangeal joint, the large toe then extends frontally and generally obliquely in the direction of the longitudinal axis 14 of the foot. The maximum length of the foot is reached in the region of the first toe, from which there is a rounded contour as far as the fifth metatarsophalangeal joint. FIG. 1 depicts the stylized contours 30, 40 of a right foot and of a left foot, respectively, with the broken lines showing the contour 40 of the left foot, and the solid lines showing the contour 30 of the right foot. The outer border shows the foot plate contour 13 of the two superposed contours 30, 40.

Arranged within the foot plate contour 13 are markings 50 which can be configured as grooves and which indicate different foot sizes both for the left foot contour and also for the right foot contour. The markings 50 on the underside of the foot plate 10 can be configured as grooves or slits or just as color markings. In order to adapt the foot plate 10 to the respective right or left foot, the material that is not needed is cut off along these markings 50. For example, if a foot plate is required for a small right foot, the material outside of the inner solid line is cut off. Markings 50 or grooves or incisions are likewise formed in the heel region 11 and can be used to adapt the length of the heel region 11. If an orthosis or an orthotic foot part is required for a large left foot, the material beyond the outermost broken-line marking 50 is cut off. In the region of the respective plantar arch there arises a common contour for all foot sizes of a foot plate 10 for a right foot or for a left foot.

In the front foot region 12, the foot plate contour 13 forms a front edge 15 which, in the illustrative embodiment shown, has two curved projections, between which a recess 16 or an incision is present in which no material of the foot plate 10 is present. The foot plate 10 can be made of a plastic, a fiber-reinforced plastic or another material. The vertex 160 is formed by the intersection of the two outer edges of the stylized contours 30, 40 for a right foot and left foot. The vertex 160 lies farther toward the heel region 11 than do the medially and laterally arranged projections or front edges of the stylized contours 30, 40 and of the foot plate contour 13. The vertex 160 lies on a line of symmetry 14, which at the same time also forms the longitudinal axis of the foot plate 10. The line of symmetry 14 is a plane of axial symmetry; the shape on both sides of the line of symmetry 14 for the orthotic foot part is the same. This affords advantages in terms of design; the adaptation to different foot shapes is effected by removing material at the edges of the foot plate 10, not by special shaping of the latter. The foot plate contour 13 flares outward from the struts 20 in the ankle region, i.e. widens out medially and laterally, in the direction of the front foot region 12, in relation to the longitudinal axis 14. In the front foot region 12, the foot plate contour 13 runs in an arc shape toward the axis of symmetry 14 and there forms a set-back region or a recess 16.

FIG. 2 shows a downwardly turned view of FIG. 1. The orthotic foot part having the foot plate 10 with the heel region 11 and the front foot region 12 provides the substantially flat foot plate 10, from which the strut 20 protrudes substantially at right angles in the proximal direction toward a lower leg or the ankle region. The strut 20 is formed in one piece with the foot plate 10 and, in the proximal end region, has a receptacle 21 for a joint device or for the rotatable mounting of a lower-leg rail. The strut 20 can be designed to be resilient in the medial-lateral direction; on account of its having a greater width than thickness, the strut 20 is more rigid in the anterior-posterior direction than in the medial-lateral direction. It is possible to produce the strut 20 from a material different than that of the foot plate 20 or to provide an insert in the mold, formed from a metal, such that the strut 20 has a metal inlay or an inlay made of a dimensionally stable material around which the material of the foot plate 10 is injected. It is thus possible to achieve permanent securing in a defined orientation perpendicular to the orientation of the foot plate 10. Alternatively, a U-shaped bracket of a structural part for forming two struts 20 can be secured reversibly and adjustably on the foot plate 10 in the region of the start of the plantar arch.

FIG. 3 shows an illustrative embodiment of the invention in a perspective view. It shows the two struts 20 which, in the region of the natural ankle joint, extend perpendicularly from the foot plate 20 in the proximal direction. The heel region 11 extends rearward beyond the struts 20, and the front foot region 12 is set back in the region of the line of symmetry 14.

FIG. 4 shows a variant of the invention in which the orthotic foot part 10, with the two struts 20 integrally formed thereon, is combined with a support element 60 in the form of a brace, which struts and support element can be coupled to each other pivotably via the receptacles 21 and 62. FIG. 4 shows an exploded view with a support element 60 not yet fitted. To fasten the latter, screws are passed through the receptacles 21 in the struts 20, and through the bores 62 arranged medially and laterally on the support element 60, and are fixed. As an alternative to pivotable fastening of the support element 60, it is possible to connect the latter rigidly to the struts 20 or just to one strut 20. The inner face of the support element 60 is provided with a padding 63, which bears on the lower leg and on the prominences of the ankle and which cushions the foot and the lower leg in relation to the struts 20 and to the support element 60. On the one hand, tolerances are compensated in this way, and, on the other hand, the need for comfort is satisfied. At the medial and lateral regions, toward the rear, the support element 60 has slits which allow a strap or buckle to be passed through, such that the support element 60 becomes a cuff which releasably encloses the lower leg. In FIG. 4, a hook 61 is arranged or integrally formed on the support element 60, on the right-hand side as seen in the lengthwise extent of the foot from rear to front, which hook 61 is hooked into an endpiece 72 of a strap 70. For this purpose, recesses or holes 71, through which the hook 61 can be guided, are arranged in the endpiece 72. The strap 70 is a device for securing the foot plate 10, together with the support element 60, to the foot and the lower leg. The strap 70 can also be elastic. By way of the recess 71 that is selected, it is possible to determine a tensile stress or a positioning of the foot plate 10 relative to the support element 60. The strap 70 can be arranged or fastened permanently on the foot plate 10, particularly in the metatarsal region or in the region of the plantar arch, when a foot is placed on the foot plate 10. The strap 70 can be arranged medially or laterally on the foot plate, depending on which contour 30, 40 is used for designing an orthosis using the orthotic foot part.

FIG. 5 shows the variant of the invention according to FIG. 4 from another perspective. Compared to the embodiment according to FIG. 4, the strap 70 via which the foot plate 10 can be held on a foot is arranged on the other side of the foot plate 10. The hook 61 on the support element 60 can be arranged or formed on both sides, i.e. laterally and medially on the support 60 element, and it is likewise possible for the hook 61 or another form-fit element to be able to be secured releasably on the medial or lateral side of the support element 60. If the foot plate according to FIG. 4 were to be designed for the right foot, the strap would be guided from the medial side of the foot plate 10 to the lateral side of the support element 60, according to FIG. 5 from the lateral side of the foot plate 10 to the medial side of the support element 60. In an embodiment of the foot plate 10 for a left foot, the reverse accordingly applies.

FIG. 6 shows, in a bottom view similar to that of FIG. 1, a variant of the invention in which the strut 20 is designed as an L-shaped bracket which, in addition to the segment extending in the vertical direction, also has a foot part segment 22 extending substantially at right angles from the strut 20 or the vertical strut segment. In this way, an L-shaped bracket is formed which is insertable into a receiving device 122 on or in the foot plate 10. The receiving device 122 can be designed as a slit or pocket into which the foot strut 22 can be inserted and can be secured on the foot plate 10. The securing can be effected reversibly, for example by a form-fit element or by a hook-and-loop fastener. It is likewise possible to effect a permanent connection, for example by welding or adhesively bonding the foot part segment 22 to the foot plate 10 in the region of the receiving device 122. With the L-shaped bracket, it is possible to obtain a medial, a lateral or a combined medial-lateral arrangement of the strut 20 on a standardized foot plate 10. By virtue of the modular configuration of the orthotic foot part with a foot plate 10 and with a separate strut body having the strut 20 and a foot part segment 22, a wide variety of orthotic foot parts can be covered without having to keep a large number of foot plates in stock. The strut 20 can be designed for different ankle heights and for different purposes of use, such that, with a limited number of struts 20, it is possible to achieve unilateral and also a bilateral routing of the support element 60, for example by superposing the foot parts segments 22 of two L-shaped brackets or else by having them abut each other in the region of the receiving device 122. It is likewise possible that the strut 20, extending in the vertical direction, is adjoined by an arranged or configured spring which, by way of a separate or integrally formed support element on the shin or on the calf, makes available a different functionality. In the case of an articulated configuration of the mounting of the support element 60 according to FIGS. 4 and 5, the joint can be guided purely at the struts 20. By way of the device 70 for securing the foot plate 10 and the orthotic foot part to the lower extremity, a dorsiflexion assist orthosis can be obtained, or malpositioning of the foot can be corrected. If the support element 60 is mounted resiliently or is connected rigidly to a resilient strut 20, an energy storage effect is obtained and walking is assisted.

1. An orthotic foot part comprising:
a foot plate with a heel region and a front foot region;
at least one strut extending in a proximal direction from the foot plate;
wherein the foot plate has a foot plate contour formed of superposed, stylized contours of a right foot and a left foot.

2. The orthotic foot part according to claim 1, wherein the foot plate contour is designed axially symmetrically with respect to a longitudinal axis, which extends from the heel region to the front foot region.

3. The orthotic foot part according to claim 1, wherein a recess oriented in the direction of the heel region is formed in a front edge of the foot plate.

4. The orthotic foot part according to claim 3, wherein the recess has a vertex lying on a line of symmetry.

5. The orthotic foot part according to claim 1, wherein markings which represent different foot sizes, both for a right foot and a left foot, are arranged on the foot plate.

6. The orthotic foot part according to claim 5, wherein the markings which represent different foot sizes are arranged in the heel region.

7. The orthotic foot part according to claim 5, wherein the markings are formed on an underside of the foot plate as depressions or grooves.

8. The orthotic foot part according to claim 1, wherein the at least one strut is designed as a spring element.

9. The orthotic foot part according to claim 1, wherein the at least one strut includes two struts arranged on the foot plate, one medially and one laterally.

10. The orthotic foot part according to claim 1, wherein the at least one strut is formed integrally on the foot plate.

11. The orthotic foot part according to claim 1, wherein the at least one strut is designed as a U-shaped or L-shaped bracket, which is secured to the foot plate.

12. The orthotic foot part according to claim 11, wherein the bracket is fastened to the foot plate in an adjustable and lockable manner.

13. The orthotic foot part according to claim 1, wherein a receptacle for a joint or a support element for a shin or calf in an applied state of the foot orthosis is arranged on the at least one strut.

14. The orthotic foot part according to claim 1, wherein a device for securing to a lower extremity is arranged on at least one of the foot plate and the strut.

15. An orthotic foot part comprising:
a foot plate comprising:
a heel region;
a front foot region;
a foot plate contour formed of superposed, stylized contours representing a right and a left foot;
at least one strut extending in a proximal direction from the foot plate.

16. Orthotic foot part according to claim 15, wherein the foot plate contour is designed axially symmetrically with respect to a longitudinal axis, which extends from the heel region to the front foot region.

17. Orthotic foot part according to claim 15, wherein a recess oriented in a direction of the heel region is formed in a front edge of the foot plate.

18. Orthotic foot part according to claim 17, wherein the recess has a vertex lying on a line of symmetry.

19. Orthotic foot part according to claim 15, wherein markings which represent different foot sizes, both for a right foot and a left foot, are arranged on the foot plate.

20. Orthotic foot part according to claim 15, wherein the markings which represent different foot sizes are arranged in the heel region.
